(19) United States
(12) Patent Application Publication
(10) Pub. No.: US 2013/0079681 A1
(43) Pub. Date: Mar. 28, 2013

(54) FOCUSED ULTRASOUND THERAPY APPARATUS AND FOCAL POINT CONTROLLING METHOD THEREOF

(71) Applicants: Ji-young Park, Yongin-si (KR); Hyoung-ki Lee, Seongnam-si (KR); Jun-ho Park, Hwaseong-si (KR)

(72) Inventors: Ji-young Park, Yongin-si (KR); Hyoung-ki Lee, Seongnam-si (KR); Jun-ho Park, Hwaseong-si (KR)

(73) Assignee: SAMSUNG ELECTRONICS CO., LTD., Suwon-si (KR)

(21) Appl. No.: 13/479,813

(22) Filed: May 24, 2012

(30) Foreign Application Priority Data
Sep. 27, 2011 (KR) 10-2011-0097568

(51) Int. Cl. A61N 7/00

(52) U.S. Cl. 601/2

(57) ABSTRACT

A focal point controlling method and a focused ultrasound therapy apparatus may be used in the treatment and removal of a lesion in a noninvasive surgical procedure by focusing ultrasonic waves. A focal point may be controlled by acquiring tissue characteristic information including physical characteristic values affecting propagation of an ultrasonic wave from a pre-diagnostic image that is a medical image obtained by capturing tissues on a path through which the ultrasonic wave propagates from a position from which the ultrasonic wave is generated to a target position on which a focal point is desired to be formed. The position at which the focal point is formed may be controlled by using the acquired tissue characteristic information.

### CROSS-REFERENCE TO RELATED APPLICATIONS

This application claims the priority benefit of Korean Patent Application No. 10-2011-0097568, filed on Sep. 27, 2011, in the Korean Intellectual Property Office, the disclosure of which is incorporated herein in its entirety by reference.

### BACKGROUND

1. Field

The present disclosure relates to a focal point controlling method of a focused ultrasound therapy apparatus that is a generally noninvasive surgery apparatus for treating and removing a lesion by focusing ultrasonic waves.

2. Description of the Related Art

Along with the development of medical science, minimally invasive surgery, and furthermore, noninvasive surgery, has recently been used for the local treatment of tumors. High Intensity Focused Ultrasound (HIFU) therapy is one noninvasive surgery method which has become widely used because it is generally harmless to a human body due to the use of a sound wave. HIFU therapy is a treatment method that causes necrosis of a lesion tissue by focusing and irradiating a high intensity ultrasonic wave on a lesion inside a human body. The ultrasonic wave is focused and irradiated on the lesion tissue and is transduced into thermal energy to increase a temperature of the irradiated part, causing coagulation necrosis in the tissue and a blood vessel. Since the temperature is instantaneously increased, only the irradiated part may be effectively removed while preventing thermal diffusion around the irradiated part.

A focused ultrasound therapy apparatus includes a transducer for transducing an electric signal into an ultrasonic wave. The focused ultrasound therapy apparatus may control a position at which a focal point is formed by adjusting a particle velocity in the transducer. To remove only a lesion without affecting surrounding tissues in focused ultrasound therapy, a focal point of an ultrasonic wave should desirably be formed on a targeted position. However, the position at which the focal point is formed may be affected by characteristics of internal tissues of a human body on an ultrasonic wave propagation path (e.g., a propagation speed of a sound wave in a tissue and a density of the tissue).

### SUMMARY

Provided is a focal point controlling method and a focused ultrasound therapy apparatus capable of controlling a fine focal point position.

Additional aspects will be set forth in part in the description which follows and, in part, will be apparent from the description, or may be learned by practice of the presented embodiments.

According to an aspect of the present invention, a focal point controlling method of a focused ultrasound therapy apparatus includes: acquiring tissue characteristic information including physical characteristic values affecting propagation of an ultrasonic wave from a pre-diagnostic image that may include a medical image which is obtained by capturing tissues on a path through which the ultrasonic wave propagates from a position from which the ultrasonic wave is generated to a target position on which a focal point is desirably or intended to be formed; and controlling the position at which the focal point is formed by using the acquired tissue characteristic information.

The acquiring of the tissue characteristic information may include acquiring tissue characteristic information of each of two or more segments obtained from the path.

The acquiring of the tissue characteristic information may include: receiving the pre-diagnostic image; calculating tissue characteristic information of the tissues on the path through which the ultrasonic wave propagates, from pre-diagnostic information acquired from the received pre-diagnostic image; segmenting the path through which the ultrasonic wave propagates into two or more segments based on the calculated tissue characteristic information; and averaging tissue characteristic information of each of the segments.

The calculating of the tissue characteristic information may include calculating the tissue characteristic information by applying the pre-diagnostic information to an experimental linear model for pre-diagnostic information and tissue characteristic information.

The calculating of the tissue characteristic information may include calculating the tissue characteristic information by using a lookup table in which tissue characteristic information corresponding to arbitrary pre-diagnostic information is stored.

The tissue characteristic information may include a propagation speed of an ultrasonic wave in a tissue and density of the tissue, and the controlling of the position at which the focal point is formed may include calculating a propagation characteristic of the ultrasonic wave on the path by using the tissue characteristic information and calculating a particle velocity for generating the ultrasonic wave at a position at which the ultrasonic wave is generated to form the focal point at the target position by using the propagation characteristic.

The propagation characteristic of the ultrasonic wave on the path may be calculated by calculating a transmission coefficient, a characteristic impedance, and a wave number of each of the segments obtained from the path by using the tissue characteristic information.

According to another aspect of the present invention, a focused ultrasound therapy apparatus for removing a lesion by irradiating an ultrasonic wave on the lesion includes: an ultrasonic transducer for generating an ultrasonic wave by transducing an electric signal; a pre-diagnostic image receiver for receiving a pre-diagnostic image from the outside, the pre-diagnostic image being, for example, a medical image obtained by capturing tissues on a path through which the ultrasonic wave propagates from a position from which the ultrasonic wave is generated to a target position on which a focal point is desirably or intended to be formed; a tissue characteristic information acquisition unit for acquiring, from the received pre-diagnostic image, tissue characteristic information including physical characteristic values affecting the propagation of the ultrasonic wave; and a focal point controller for controlling the ultrasonic transducer to form the focal point on the target position by using the acquired tissue characteristic information.

The tissue characteristic information acquisition unit may acquire tissue characteristic information of each of two or more segments obtained from the path.

The tissue characteristic information acquisition unit may include: a pre-diagnostic information acquisition unit for acquiring pre-diagnostic information from the received pre-diagnostic image; a path segmentation unit for segmenting the path into a plurality of segments based on the acquired pre-diagnostic information; a segment average value calculator for averaging pre-diagnostic information of each of the segments; and a tissue characteristic information mapping unit for mapping the averaged pre-diagnostic information to tissue characteristic information by using a lookup table.

The tissue characteristic information may include a propagation speed of an ultrasonic wave in a tissue and density of the tissue, and the focal point controller may include: a beamforming algorithm execution unit for calculating a propagation characteristic of the ultrasonic wave on the path by using the tissue characteristic information received from the tissue characteristic information acquisition unit and calculating a particle velocity in the ultrasonic transducer to form the focal point at the target position by using the propagation characteristic; and a particle velocity controller for controlling the ultrasonic transducer based on the calculated particle velocity.

The beamforming algorithm execution unit may calculate a transmission coefficient, a characteristic impedance, and a wave number of each of the segments obtained from the path by using the tissue characteristic information received from tissue characteristic information acquisition unit and calculate a propagation characteristic of the ultrasonic wave on the path by using the transmission coefficient, the characteristic impedance, and the wave number of each of the segments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram of a focused ultrasound therapy apparatus according to an embodiment of the present invention;

FIGS. 2 and 3 are detailed block diagrams of a tissue characteristic information acquisition unit of the focused ultrasound therapy apparatus of FIG. 1, according to embodiments of the present invention;

FIG. 4 is a detailed block diagram of a focal point controller of the focused ultrasound therapy apparatus FIG. 1, according to an embodiment of the present invention;

FIGS. 5 and 6 illustrate propagation paths of an ultrasonic wave that are segmented into a plurality of segments; and

FIGS. 7 to 10 are flowcharts illustrating a focal point controlling method of a focused ultrasound therapy apparatus, according to embodiments of the present invention.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. To more clearly describe characteristics of the embodiments, well-known functions or constructions in the technical field to which the embodiments belong are not described in detail.

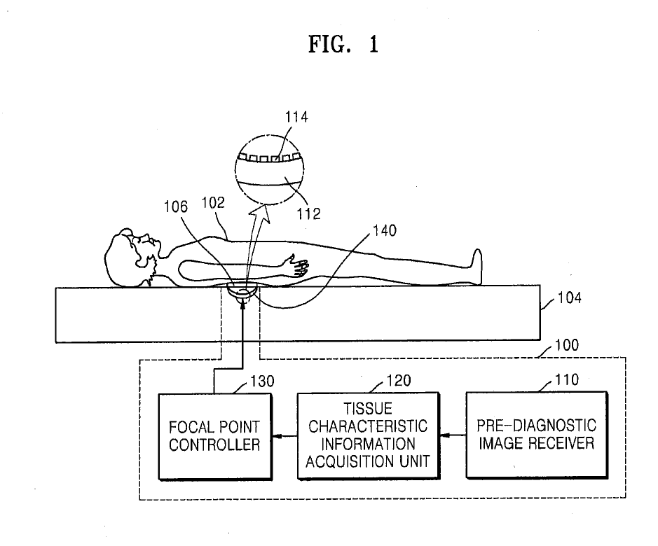

FIG. 1 is a block diagram of a focused ultrasound therapy apparatus 100 according to an embodiment of the present invention. Referring to FIG. 1, the focused ultrasound therapy apparatus 100 may include a pre-diagnostic image receiver 110, a tissue characteristic information acquisition unit 120, a focal point controller 130, and an ultrasonic transducer 140.

As shown in FIG. 1, the ultrasonic transducer 140 may be installed inside a bed 104 on which a subject 102 to be diagnosed lays to remove a lesion by irradiating an ultrasonic wave on a predetermined part of a body of the subject 102. Here, a gel pad 106 to help the traveling of the ultrasonic wave may be disposed between the subject 102 and the bed 104. In addition, as shown in a magnified figure in FIG. 1, the ultrasonic transducer 140 may include a plurality of elements 114 for generating the ultrasonic wave on a round-type support plate 112 sunken at the center. By controlling a magnitude and phase of a particle velocity in each of the plurality of elements 114, a position at which a focal point of the ultrasonic wave generated by the plurality of elements 114 is formed may be controlled. Since the ultrasonic transducer 140 is generally used in focused ultrasound therapy apparatuses, a detailed description thereof is omitted herein. Hereinafter, a detailed operation of the focused ultrasound therapy apparatus 100 is described.

The pre-diagnostic image receiver 110 receives a pre-diagnostic image from an external source. The pre-diagnostic image may include a medical image, e.g., a Computed Tomography (CT) image or a Magnetic Resonance Imaging (MRI) image, obtained by capturing tissues around a lesion to be removed before performing treatment with the focused ultrasound therapy apparatus 100. In particular, the pre-diagnostic image may be an image obtained by capturing tissues from a position from which the ultrasonic wave is generated (e.g., the ultrasonic transducer 140), to a position of the lesion to be removed (e.g., a target position on which the focal point is formed).

The pre-diagnostic image includes physical information of the body's internal tissues around the lesion to be removed. For example, a CT image is represented by a relative value of a CT number obtained by substituting a linear absorption coefficient of a tissue perceived by a CT machine into Equation 1. The linear absorption coefficient indicates an absorption degree of an X-ray when the X-ray travels 1 cm in a material, and a unit thereof is cm⁻¹. In Equation 1, μ denotes a linear absorption coefficient of a captured tissue, and μ_{W} denotes a linear absorption coefficient of water, and K is a constant number, for example, an integer constant.

$$CT number = K \frac{μ - {μ}_{W}}{{μ}_{W}} \qquad ( 1 )$$

Information, such as a CT number, regarding physical characteristics of a tissue that is obtained from a pre-diagnostic image may be considered as an example of pre-diagnostic information.

The tissue characteristic information acquisition unit 120 acquires pre-diagnostic information from the pre-diagnostic image received from the pre-diagnostic image receiver 110 and then acquires tissue characteristic information of internal tissues on a path through which the ultrasonic wave propagates by using the acquired pre-diagnostic information. The tissue characteristic information may include, but is not limited to, physical characteristic values of a tissue affecting propagation of an ultrasonic wave, e.g., a propagation speed c of the ultrasonic wave in the tissue, density ρ of the tissue, and an acoustic impedance Z. Table 1 shows CT numbers, ultrasonic wave propagation speeds c, tissue densities ρ, and acoustic impedances Z of various tissues.

TABLE 1

| Material | CT number | p(kg/m3) | c(m/s) | Z(kg/(mm2s)) |
|---|---|---|---|---|
| Bone | 1000 | 1912 | 4080 | 7.8 |
| Muscle | 10~40 | 1080 | 1580 | 1.7 |
| Liver | 40~60 | 1060 | 1550 | 1.64 |
| Blood | 40 | 1057 | 1575 | 1.62 |
| Kidney | 30 | 1038 | 1560 | 1.62 |
| Brain | 43~46 | 994 | 1560 | 1.55 |
| Water | 0 | 988 | 1497 | 1.48 |
| Fat | −100~−50 | 952 | 1459 | 1.38 |
| Air | −1000 | 1.2 | 330 | 0.0004 |

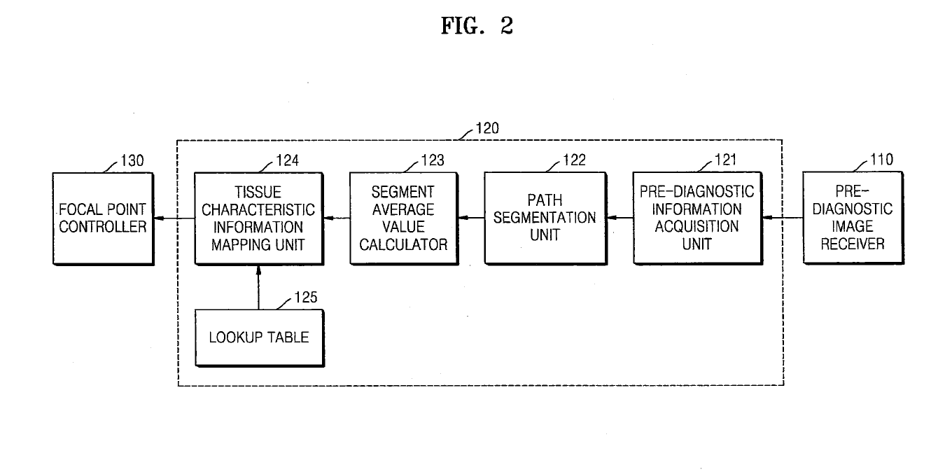

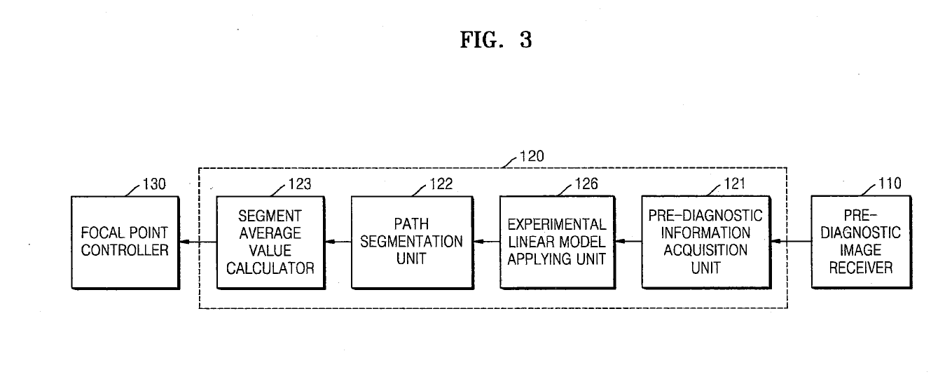

The tissue characteristic information acquisition unit 120 may acquire the tissue characteristic information by using the pre-diagnostic information. Methods of acquiring tissue characteristic information from pre-diagnostic information include a method using an experimental linear model of pre-diagnostic information and tissue characteristic information and a method using a lookup table that stores tissue characteristic information corresponding to arbitrary pre-diagnostic information. These methods will be described in detail in the description of FIGS. 2 and 3 in which a detailed configuration of the tissue characteristic information acquisition unit 120 is shown.

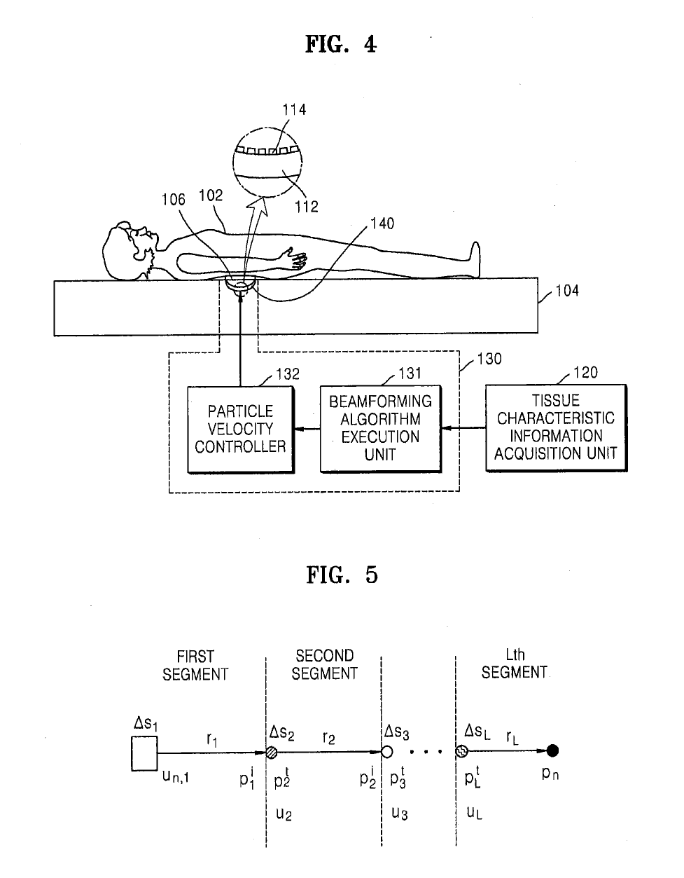

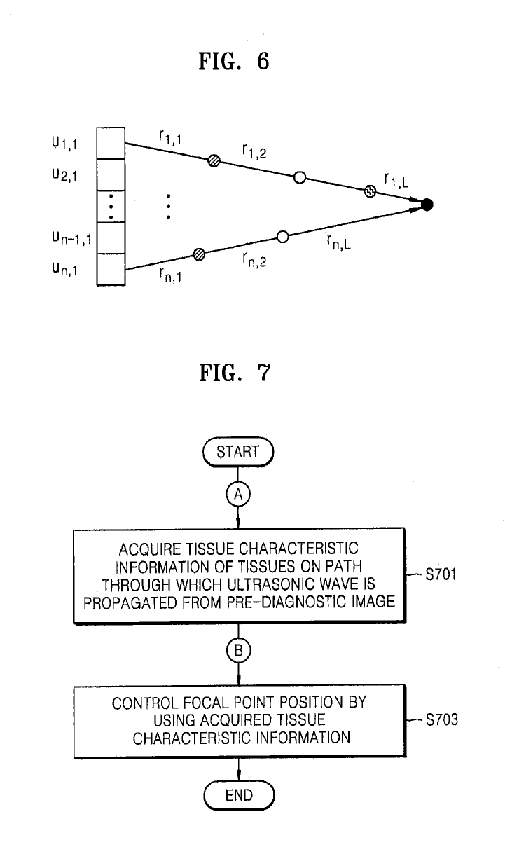

The focal point controller 130 controls the position at which the focal point of the ultrasonic wave is formed by using the tissue characteristic information acquired by the tissue characteristic information acquisition unit 120. The characteristics of the internal tissues on the path through which the ultrasonic wave propagates from the position from which the ultrasonic wave is generated to the position at which the focal point is formed is generally inhomogeneous. However, there is a problem that a position at which an actual focal point is formed is quite different from a target position at which a focal point is supposed to be originally formed because a propagation speed of an ultrasonic wave is set as 1540 m/s, a representative value of a soft tissue, under an assumption that the body's internal tissues are homogeneous in a focal point control. According to the current embodiment, since tissue characteristic information acquired from a pre-diagnostic image reflects an inhomogeneous characteristic of the body's internal tissues, a relatively correct (i.e., more precise) focal point control may be achieved by using the tissue characteristic information. A method of controlling a focal point by reflecting inhomogeneous tissue characteristic information will be described in detail in the description of FIG. 4, showing a detailed configuration of the focal point controller 130 with reference to FIGS. 5 and 6. Prior to the description of the method of controlling a focal point by reflecting inhomogeneous tissue characteristic information, a beamforming algorithm for controlling a focal point in the case of a homogeneous tissue characteristic is schematically described with equations.

If it is assumed that the ultrasonic transducer 140 includes N elements 114 in a row and focal points are formed at M target positions, a pressure p applied to an mth (m=1, 2, . . . , M) target position by the N elements may be obtained by a Rayleigh-Sommerfeld integral expressed by Equation 2.

$$\frac{j ρ ck}{2 π} {\sum }_{n = 1}^{N} {u}_{n} {\int }_{{S}_{n}}^{ } \frac{{e}^{- j k \vert {r}_{m} - {r}_{n} \vert}}{\vert {r}_{m} - {r}_{n} \vert} d {S}_{n} = p ( {r}_{m} ) \qquad ( 2 )$$

In Equation 2, ρ, c, and k denote density of a homogeneous tissue, a propagation speed of an ultrasonic wave in the tissue, and a wave number, respectively; rₙ and rₘ denote an nth (n=1, 2, . . . , N) element and a position vector of the mth target position, respectively; Sn denotes a cross section of the nth element, uₙ denotes a particle velocity at the nth element, and p(rₘ) denotes a pressure at the mth target position having the position vector rₘ. From Equation 2, a relational expression between the particle velocity at the nth element and the pressure applied to the mth target position, i.e., an ultrasonic wave propagation characteristic, may be obtained by Equation 3.

$$H ( m , n ) = \frac{j ρ ck}{2 π} {\int }_{{S}_{n}}^{ } \frac{{e}^{- j k \vert {r}_{m} - {r}_{n} \vert}}{\vert {r}_{m} - {r}_{n} \vert} d {S}_{n} \qquad ( 3 )$$

Equation 4 describes a relational expression among a matrix u of particle velocities at the n elements, a matrix p of pressures applied to the m target positions, and an ultrasonic wave propagation characteristic matrix H, is derived from Equations 2 and 3. Thus, a particle velocity at each element may be obtained by a pseudo-inverse method expressed by Equations 5 and 6.

Hu=p (4)

u=H⁺p (5)

u=H*^{t}(HH*^{t})⁻¹p (6)

In Equations 5 and 6, H⁺ denotes a pseudo-inverse matrix of H, and H*^{t} denotes a conjugate transpose matrix of H. A method of applying the above-described beamforming algorithm for controlling a focal point in a homogeneous tissue to an inhomogeneous tissue will be described in detail in the description of FIGS. 4, 5, and 6.

By using the focused ultrasound therapy apparatus 100, a relatively correct focal point may be controlled by acquiring tissue characteristic information of the body's inhomogeneous internal tissues on a path through which an ultrasonic wave propagates based on a pre-diagnostic image and through controlling the focal point based on the acquired tissue characteristic information.

Hereinafter, embodiments based on the embodiment shown in FIG. 1 are described in more detail with reference to FIGS. 2 to 6. For ease of understanding, it is assumed that a pre-diagnostic image is a CT image and tissue characteristic information includes a propagation speed c of an ultrasonic wave in a tissue and density ρ of the tissue.

FIG. 2 is a detailed block diagram of the tissue characteristic information acquisition unit 120 of the focused ultrasound therapy apparatus 100 of FIG. 1, according to an embodiment of the present invention. Referring to FIG. 2, the tissue characteristic information acquisition unit 120 may include a pre-diagnostic information acquisition unit 121, a path segmentation unit 122, a segment average value calculator 123, a tissue characteristic information mapping unit 124, and a lookup table 125. The pre-diagnostic information acquisition unit 121 acquires CT numbers from a CT image received from the pre-diagnostic image receiver 110. The pre-diagnostic information may include information regarding physical characteristics of a tissue obtained from a pre-diagnostic image, and in the case of the CT image, the CT numbers correspond to the pre-diagnostic information. The path segmentation unit 122 segments a path through which an ultrasonic wave propagates into a plurality of segments based on the acquired CT numbers. For example, if there is a position at which a CT number suddenly changes, both sides of the path divided by setting the position as a boundary may be segmented as two segments. Optionally, the path through which the ultrasonic wave propagates may be segmented into a plurality of segments having the same length. As another option, the path may be segmented according to a user's input. As described above, a method of segmenting the path may be variously implemented without being limited to one, and may include more than one method, or a combination thereof.

When the path through which the ultrasonic wave propagates is segmented into a plurality of segments, the segment average value calculator 123 calculates a CT number average value of a path corresponding to each of the segments. Thereafter, the tissue characteristic information mapping unit 124 maps a CT number average value of each of the segments to a corresponding ultrasonic wave propagation speed c and tissue density ρ, and in this case, the lookup table 125 that stores ultrasonic wave propagation speeds c and tissue densities p corresponding to arbitrary CT numbers may be used. Finally, the tissue characteristic information mapping unit 124 transmits to the focal point controller 130 tissue characteristic information, which may include an ultrasonic wave propagation speed c and tissue density ρ, of each of the segments obtained from the path through which the ultrasonic wave propagates.

FIG. 3 is a detailed block diagram of the tissue characteristic information acquisition unit 120 of the focused ultrasound therapy apparatus 100 of FIG. 1, according to another embodiment of the present invention. Referring to FIG. 3, the tissue characteristic information acquisition unit 120 according to the current embodiment may include the pre-diagnostic information acquisition unit 121, an experimental linear model applying unit 126, the path segmentation unit 122, and the segment average value calculator 123. Once the pre-diagnostic information acquisition unit 121 acquires CT numbers from a CT image, the experimental linear model applying unit 126 may obtain ultrasonic wave propagation speeds c and tissue densities ρ by applying the acquired CT numbers to an experimental linear model. There is an almost linear characteristic in a soft tissue between CT numbers of a tissue and corresponding ultrasonic wave propagation speeds c in the tissue and tissue densities ρ, and thus, an experimental linear model may be made by using experimentally measured values. For example, an experimental linear model expressed by Equations 7 and 8 may be made. In Equations 7 and 8, h denotes a CT number. Of course, various experimental linear models may be made from measurement results and making an experimental model is not limited to Equations 7 and 8.

c=0.8648h+1540.5024 (7)

p=0.9489h+1025.4206 (8)

By calculating ultrasonic wave propagation speeds c and tissue densities p from CT numbers by using a pre-made experimental linear model, tissue characteristic information approximate to actual values may be conveniently obtained from a pre-diagnostic image even without directly measuring actual tissue characteristic information of each tissue.

The path segmentation unit 122 segments a path through which an ultrasonic wave propagates into a plurality of segments based on the ultrasonic wave propagation speeds c and the tissue densities p calculated by the experimental linear model applying unit 126. For example, if there is a position at which an ultrasonic wave propagation speed c and a tissue density ρ suddenly change, both sides of the path divided by setting the position as a boundary may be segmented as two segments. Optionally, the segmentation may be performed by setting the boundary as a position at which any one of the ultrasonic wave propagation speed c and the tissue density ρ suddenly changes. As another option, the path through which the ultrasonic wave propagates may be segmented into a plurality of segments having the same length. As another option, the path may be segmented according to a user's input. As described above, a method of segmenting the path may be variously implemented without being limited to one, and may include more than one method, or a combination thereof. When the path through which the ultrasonic wave propagates is segmented into a plurality of segments, the segment average value calculator 123 calculates an average value of the ultrasonic wave propagation speeds c and an average value of the tissue densities p for a path corresponding to each of the segments and transmits the calculated average values to the focal point controller 130.

FIG. 4 is a detailed block diagram of the focal point controller 130 of the focused ultrasound therapy apparatus 100 of FIG. 1, according to an embodiment of the present invention. Referring to FIG. 4, the focal point controller 130 may include a beamforming algorithm execution unit 131 and a particle velocity controller 132. The beamforming algorithm execution unit 131 executes a beamforming algorithm by using the tissue characteristic information received from the tissue characteristic information acquisition unit 120. Hereinafter, a method of performing a beamforming algorithm by using tissue characteristic information of an inhomogeneous tissue is described in detail with reference to FIGS. 5 and 6 and equations.

FIG. 5 illustrates a propagation path of an ultrasonic wave segmented into a plurality of segments. If it is assumed that a plurality of elements are arranged in the ultrasonic transducer 140 in a matrix pattern, a pressure applied to a target position by an ultrasonic wave generated at a particle velocity u_{n,1} by an nth element of a first row is pₙ. As illustrated in FIG. 5, p₁ⁱ and p₂^{t} denote a pressure applied from a first segment to a boundary between the first segment and a second segment and a pressure applied from the boundary between the first segment and the second segment to the second segment, respectively; u₂ and u₃ denote a particle velocity transferred from the boundary between the first segment and the second segment to the second segment and a particle velocity transferred from a boundary between the second segment and a third segment to the third segment, respectively; Δs indicates a cross section to where a pressure is applied; and r₁ denotes a distance between the element and the boundary between the first segment and the second segment. In FIG. 5, a path through which the ultrasonic wave propagates from the element to the target position is segmented into L segments.

A transmission coefficient T, a characteristic impedance Z, and a wave number k of each segment may be obtained from an ultrasonic wave propagation speed c and a tissue density ρ. For example, a transmission coefficient T₁, a characteristic impedance Z₁, and a wave number k₁ of the first segment may be obtained from Equation 9.

$${T}_{1} = \frac{2 {Z}_{2}}{{Z}_{1} + {Z}_{2}} , \\ {Z}_{1} = {ρ}_{1} {c}_{1} , \\ {k}_{1} = \frac{ω}{{c}_{1}} \qquad ( 9 )$$

The following equations may be derived by using values obtained from Equation 9.

$${p}_{1}^{i} = \frac{j {Z}_{1} {k}_{1}}{2 π} \frac{{e}^{- j {k}_{1} {r}_{1}}}{{r}_{1}} Δ {s}_{1} {u}_{n , 1} \qquad ( 10 )$$

The pressure p₁ⁱ applied from the first segment to the boundary between the first segment and the second segment is obtained from the particle velocity u_{n,1} at the element by using the characteristic impedance Z₁ of the first segment.

p₂^{t}=p₁ⁱT₁=Z₂u₂ (11)

The pressure p₂^{t} obtained by using p₁ⁱ and T₁ may be expressed by multiplication of a characteristic impedance Z₁ of the second segment by a particle velocity u₂ transferred from the boundary between the first segment and the second segment to the second segment.

$${u}_{2} = \frac{j {k}_{1}}{2 π} \frac{{e}^{- j {k}_{1} {r}_{1}}}{{r}_{1}} \frac{{Z}_{1} {T}_{1}}{{Z}_{2}} Δ {s}_{1} {u}_{n , 1} \qquad ( 12 )$$

By rearranging Equations 10 and 11, a relational expression between u₂ and u_{n,1} may be obtained as expressed by Equation 12. By repeating the above process for the second to Lth segments, a relational expression between pₙ and u_{n,1} may be obtained as expressed by the following equations. The detailed process is expressed by Equations 13 to 19.

$$\begin{matrix}{p}_{2}^{i} = \frac{j {Z}_{2} {k}_{2}}{2 π} \frac{{e}^{- j {k}_{2} {r}_{2}}}{{r}_{2}} Δ {s}_{2} {u}_{2} & ( 13 ) \\ {p}_{3}^{t} = {p}_{2}^{i} {T}_{2} = {Z}_{3} {u}_{3} & ( 14 ) \\ {u}_{3} = {p}_{2}^{i} \frac{{T}_{2}}{{Z}_{3}} = \frac{j {k}_{2}}{2 π} \frac{{e}^{- j {k}_{2} {r}_{2}}}{{r}_{2}} \frac{{Z}_{2} {T}_{2}}{{Z}_{3}} Δ {s}_{2} {u}_{2} & ( 15 ) \\ {u}_{3} = [ {\prod }_{l = 1}^{2} ( \frac{j {k}_{l}}{2 π} \frac{{e}^{- j {k}_{l} {r}_{l}}}{{r}_{l}} \frac{{Z}_{l} {T}_{l}}{{Z}_{l + 1}} ) Δ {s}_{l} ] {u}_{n , 1} & ( 16 )\end{matrix}$$

Equation 16 is a relational expression between u₃ and u_{n,1} which may be obtained from Equations 13, 14, and 15. When Equation 16 is isolated for u_{L}, a relational expression between u_{L} and u_{n,1} may be obtained as expressed by Equation 17.

$${u}_{L} = [ {\prod }_{l = 1}^{L - 1} ( \frac{j {k}_{l}}{2 π} \frac{{e}^{- j {k}_{l} {r}_{l}}}{{r}_{l}} \frac{{Z}_{l} {T}_{l}}{{Z}_{l + 1}} ) Δ {s}_{l} ] {u}_{n , 1} \qquad ( 17 )$$

Equation 18 may further be obtained from Equation 16.

$${p}_{n} = \frac{j {Z}_{L} {k}_{L}}{2 π} \frac{{e}^{- j {k}_{L} {r}_{L}}}{{r}_{L}} Δ {s}_{L} {u}_{L} \qquad ( 18 )$$

Thus, the relational expression between pₙ and u_{n,1} may finally be obtained as expressed by Equation 19 by using Equations 17 and 18.

$${p}_{n} = ( \frac{j {Z}_{L} {k}_{L}}{2 π} \frac{{e}^{- j {k}_{L} {r}_{L}}}{{r}_{L}} Δ {s}_{L} ) [ {\prod }_{l = 1}^{L - 1} ( \frac{j {k}_{l}}{2 π} \frac{{e}^{- j {k}_{l} {r}_{l}}}{{r}_{l}} \frac{{Z}_{l} {T}_{l}}{{Z}_{l + 1}} ) Δ {s}_{l} ] {u}_{n , 1} \qquad ( 19 )$$

FIG. 6 illustrates n elements arranged in a row. If it is assumed that a pressure applied to a target position by a particle velocity u_{n,1} of an nth element is pₙ and an ultrasonic wave propagation characteristic on a path is Hₙ, the ultrasonic wave propagation characteristic Hₙ as expressed by Equation 21 may be obtained from Equation 20 by using a characteristic impedance and a wave number of each segment.

$$\begin{matrix}{p}_{n} = {H}_{n} {u}_{n , 1} & ( 20 ) \\ {H}_{n} = ( \frac{j {Z}_{L} {k}_{L}}{2 π} \frac{{e}^{- j {k}_{L} {r}_{n , L}}}{{r}_{n , L}} Δ {s}_{L} ) [ {\prod }_{l = 1}^{L - 1} ( \frac{j {k}_{l}}{2 π} \frac{{e}^{- j {k}_{l} {r}_{n , l}}}{{r}_{n , l}} \frac{{Z}_{l} {T}_{l}}{{Z}_{l + 1}} ) Δ {s}_{l} ] & ( 21 )\end{matrix}$$

That is, the ultrasonic wave propagation characteristic Hₙ may be obtained by using the tissue characteristic information (an ultrasonic wave propagation speed and tissue density) of each segment of the path through which the ultrasonic wave propagates that is received from the tissue characteristic information acquisition unit 120.

p=p₁+p₂+ . . . +pₙ, H=[H₁ H₂ . . . Hₙ], u=[u_{1,1} u_{2,1} . . . u_{n,1}] (22)

Hu=p (23)

u=H⁺p (24)

As expressed by Equations 22, 23, and 24, a particle velocity u at an element may be obtained by the pseudo-inverse method even in an inhomogeneous tissue as well as in a homogeneous tissue. The beamforming algorithm execution unit 131 transmits a particle velocity at each element obtained by performing and applying the beamforming algorithm described above to the particle velocity controller 132. The particle velocity controller 132 controls a particle velocity at each element of the ultrasonic transducer 140 based on the received particle velocity.

As described above, by segmenting a path through which an ultrasonic wave propagates into a plurality of segments and performing the beamforming algorithm with tissue characteristic information of each of the segments, a correct focal point may be controlled even when tissues on the path are inhomogeneous.

FIGS. 7 to 10 are flowcharts illustrating a focal point controlling method of a focused ultrasound therapy apparatus, according to embodiments of the present invention. Hereinafter, the focal point controlling method of the focused ultrasound therapy apparatus is described in detail with reference to FIGS. 7 to 10.

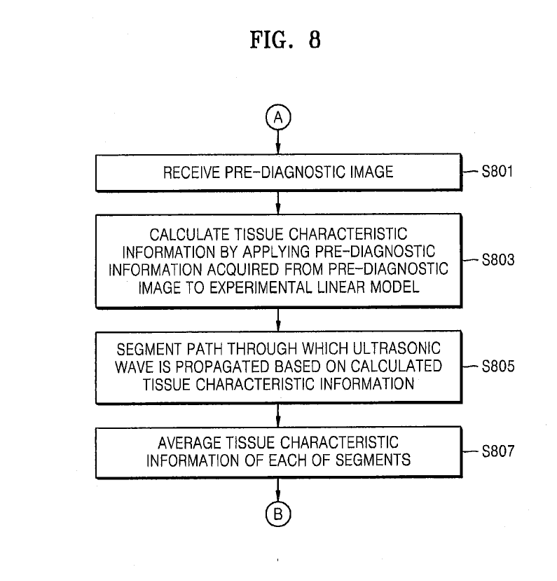

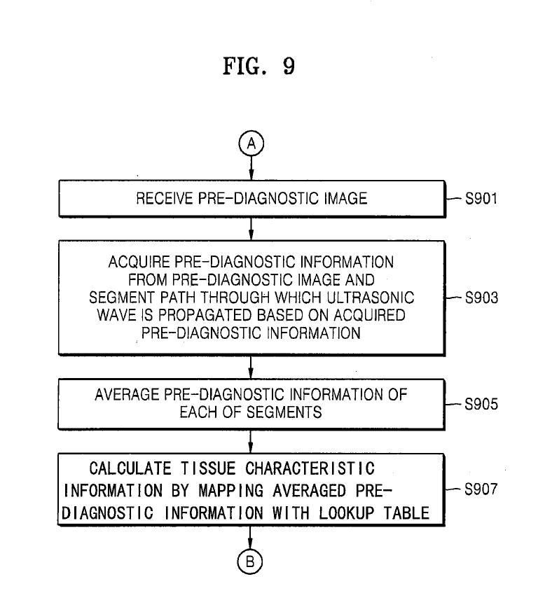

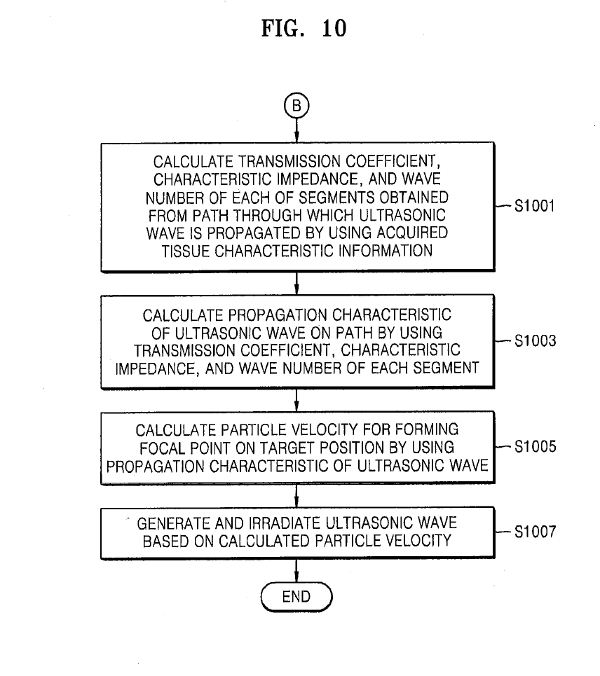

Referring to FIG. 7, in operation S701, tissue characteristic information of tissues on a path through which an ultrasonic wave propagates is acquired from a pre-diagnostic image. For example, when the pre-diagnostic image is a CT image, CT numbers are acquired from the CT image, and tissue characteristic information, such as ultrasonic wave propagation speeds and tissue densities, in the tissues on the path through which an ultrasonic wave propagates is acquired by using the acquired CT numbers. An embodiment of acquiring tissue characteristic information from a pre-diagnostic image will be described in detail in the description of FIGS. 8 and 9, which is a detailed description of operation S701. In operation S703, a focal point is controlled by using the acquired tissue characteristic information. An embodiment of operation S703 will be described in detail with reference to FIG. 10 below. According to the current embodiment, by acquiring, from a pre-diagnostic image, tissue characteristic information of tissues on a path through which an ultrasonic wave propagates, the tissue characteristic information may be conveniently acquired without measuring internal tissues of a human body one-by-one. In addition, by controlling a focal point position by using the tissue characteristic information acquired from the pre-diagnostic image, an error of the focal point position that may occur when tissues on the path are inhomogeneous may be reduced.

FIGS. 8 and 9 are flowcharts illustrating operation S701 according to embodiments of the present invention. Referring to FIG. 8, in operation S801, a pre-diagnostic image is received. In operation S803, pre-diagnostic information is acquired from the received pre-diagnostic image, and tissue characteristic information is acquired by applying the acquired pre-diagnostic information to an experimental linear model associated with pre-diagnostic information and tissue characteristic information. For example, when the pre-diagnostic image is a CT image, tissue characteristic information, such as ultrasonic wave propagation speeds c in a tissue and tissue densities ρ, is calculated by receiving the CT image and applying CT numbers acquired from the CT image to the experimental linear model. There is an almost linear characteristic in a soft tissue between CT numbers of a tissue and corresponding ultrasonic wave propagation speeds c in the tissue and tissue densities ρ, and thus, an experimental linear model may be made by using experimentally measured values.

In operation S805, a path through which an ultrasonic wave propagates is segmented based on the calculated tissue characteristic information. That is, the path through which the ultrasonic wave propagates is segmented into a plurality of segments based on the calculated ultrasonic wave propagation speeds c and tissue densities ρ. For example, if there is a position at which a CT number suddenly changes, both sides of the path divided by setting the position as a boundary may be segmented as two segments. Optionally, the segmentation may be performed by setting the boundary as a position at which any one of an ultrasonic wave propagation speed c and tissue density ρ suddenly changes. As another option, the path through which the ultrasonic wave propagates may be segmented into a plurality of segments having the same length. As another option, the path may be segmented according to a user's input. As described above, a method of segmenting the path may be variously implemented without being limited to one, and may include more than one method, or a combination thereof. When the path is segmented into a plurality of segments in operation S805, a representative value of each segment is calculated by averaging the tissue characteristic information for each of the segments in operation S807.

Referring to FIG. 9, in operation S901, a pre-diagnostic image is received. In operation S903, pre-diagnostic information is acquired from the received pre-diagnostic image, and a path through which an ultrasonic wave propagates is segmented based on the acquired pre-diagnostic information. For example, when the pre-diagnostic image is a CT image, the CT image is received and the path is segmented based on CT numbers acquired from the received CT image. For example, if there is a position at which a CT number suddenly changes, both sides of the path divided by setting the position as a boundary may be segmented as two segments. Optionally, the path through which the ultrasonic wave propagates may be segmented into a plurality of segments having the same length. As another option, the path may be segmented according to a user's input. As described above, a method of segmenting the path may be variously implemented without being limited to one, and may include more than one method, or a combination thereof.

In operation S905, the pre-diagnostic information in each of the segments is averaged. That is, CT numbers in each segment are averaged, and the average value of each segment is obtained as a representative value of each corresponding segment. In operation S907, tissue characteristic information is obtained by mapping the pre-diagnostic information averaged for each segment with a lookup table in which tissue characteristic information corresponding to arbitrary pre-diagnostic information is stored.

According to the embodiments shown in FIGS. 8 and 9, by segmenting a path through which an ultrasonic wave propagates into a plurality of segments based on pre-diagnostic information or tissue characteristic information and acquiring a representative value of tissue characteristic information of each segment by an averaging operation, a focal point may be effectively controlled even when tissues on the path are inhomogeneous.

FIG. 10 is a flowchart illustrating operation S703 of FIG. 7, according to an embodiment of the present invention. Referring to FIG. 10, in operation S1001, a transmission coefficient, a characteristic impedance, and a wave number of each of the segments obtained from a path through which an ultrasonic wave propagates are calculated by using tissue characteristic information. A detailed method of calculating the transmission coefficient, the characteristic impedance, and the wave number from the tissue characteristic information is referred to from the description of FIGS. 4 to 6 and Equation 9. In operation S1003, a propagation characteristic of the ultrasonic wave on the path may be obtained by using the transmission coefficient, the characteristic impedance, and the wave number of each segment. A detailed method of obtaining the propagation characteristic of the ultrasonic wave on the path is referred to from the description of Equations 10 to 21. In operation S1005, a particle velocity for forming a focal point on a target position by using the ultrasonic wave propagation characteristic is calculated in operation S1003. A detailed method of calculating the particle velocity is referred to from the description of Equations 22 to 24. When the particle velocity is calculated, an ultrasonic wave is generated and irradiated according to the calculated particle velocity in operation S1007.

As described above, by segmenting a path through which an ultrasonic wave propagates into a plurality of segments and performing the beamforming algorithm with tissue characteristic information of each of the segments, a correct focal point may be controlled even when tissues on the path are inhomogeneous.

As described above, according to the one or more of the above embodiments of the present invention, by acquiring, from a pre-diagnostic image, tissue characteristic information of tissues on a path through which an ultrasonic wave propagates, the tissue characteristic information may be conveniently acquired without measuring internal tissues of a human body one-by-one. In addition, by controlling a focal point position by using the tissue characteristic information acquired from the pre-diagnostic image, an error of the focal point position that may occur when tissues on the path are inhomogeneous may be reduced.

It should be understood that the exemplary embodiments described herein should be considered in a descriptive sense only and not for purposes of limitation. Descriptions of features or aspects within each embodiment should typically be considered as available for other similar features or aspects in other embodiments.

For example, the disclosure has described one or more embodiments in which a focused ultrasound therapy apparatus and focal controlling method may be used to treat humans. However, the focused ultrasound therapy apparatus and focal controlling method may be used in the treatment of other life forms, including animals. Additionally, it should be noted that while FIGS. 1 and 4 illustrate treatment of a lesion using an ultrasonic transducer while a subject lays on a bed, the disclosure is not so limited. For example, the subject may be treated while in another position, the ultrasonic transducer may be disposed elsewhere to treat another area of the subject, there may not be a bed, or there may be another object in which the ultrasonic transducer is installed.

In one or more previously described embodiments, it has been disclosed that the pre-diagnostic image receiver may receive a pre-diagnostic image from an external source. For example, the pre-diagnostic image may be obtained via a wired or wireless network, or from a non-transitory computer-readable media including magnetic media such as hard disks, floppy disks, and magnetic tape; optical media such as CD ROM disks and DVDs; magneto-optical media such as optical disks; or from other hardware devices that are configured to store data.

In one or more previously described embodiments, it has also been disclosed that the a representative value of a segment may be calculated using average tissue characteristic information values for each of the segments, or averaging CT numbers in each segment. However, the disclosure is not so limited. For example, one of ordinary skill in the art would appreciate that other statistical approaches may be utilized, such as using a median value instead of an average value.

The terms “module”, and “unit,” as used herein, may refer to, but is not limited to, a software or hardware component or device, such as a Field Programmable Gate Array (FPGA) or Application Specific Integrated Circuit (ASIC), which performs certain tasks. A module or unit may be configured to reside on an addressable storage medium and configured to execute on one or more processors. Thus, a module or unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules/units may be combined into fewer components and modules/units or further separated into additional components and modules.

The focused ultrasound therapy apparatus and focal controlling method according to the above-described example embodiments may use one or more processors, which may include a microprocessor, central processing unit (CPU), digital signal processor (DSP), or application-specific integrated circuit (ASIC), as well as portions or combinations of these and other processing devices.

The focal controlling method according to the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules to perform the operations of the above-described example embodiments, or vice versa.

Thus, as set forth above, although a few embodiments have been shown and described, it would be appreciated by those skilled in the art that changes may be made in these embodiments without departing from the principles and spirit of the invention, the scope of which is defined by the claims and their equivalents.

What is claimed is:

1. A focal point controlling method using a focused ultrasound therapy apparatus by focusing and irradiating an ultrasonic wave, the focal point controlling method comprising:
acquiring tissue characteristic information including physical characteristic values affecting propagation of an ultrasonic wave using a pre-diagnostic image which includes tissue characteristic information of tissues on a path through which the ultrasonic wave propagates from a position from which the ultrasonic wave is generated to a target position on which a focal point is to be formed; and
controlling the target position at which the focal point is to be formed by using the acquired tissue characteristic information.

2. The focal point controlling method of claim 1, wherein the acquiring of the tissue characteristic information comprises acquiring tissue characteristic information of each of two or more segments obtained from the path.

3. The focal point controlling method of claim 2, wherein the acquiring of the tissue characteristic information comprises:
receiving the pre-diagnostic image;
calculating tissue characteristic information of the tissues on the path through which the ultrasonic wave propagates, from pre-diagnostic information acquired from the received pre-diagnostic image;
segmenting the path through which the ultrasonic wave propagates into two or more segments based on the calculated tissue characteristic information; and
averaging tissue characteristic information of each of the segments.

4. The focal point controlling method of claim 3, wherein the calculating of the tissue characteristic information comprises calculating the tissue characteristic information by applying the pre-diagnostic information to an experimental linear model.

5. The focal point controlling method of claim 3, wherein the calculating of the tissue characteristic information comprises calculating the tissue characteristic information by using a lookup table in which tissue characteristic information corresponding to arbitrary pre-diagnostic information is stored.

6. The focal point controlling method of claim 2, wherein the acquiring of the tissue characteristic information comprises:
receiving the pre-diagnostic image;
segmenting the path through which the ultrasonic wave propagates into two or more segments based on pre-diagnostic information acquired from the received pre-diagnostic image;
averaging the pre-diagnostic information for each of the segments; and
calculating tissue characteristic information from the pre-diagnostic information averaged for each of the segments.

7. The focal point controlling method of claim 6, wherein the calculating of the tissue characteristic information comprises calculating the tissue characteristic information by applying the pre-diagnostic information averaged for each of the segments to an experimental linear model.

8. The focal point controlling method of claim 6, wherein the calculating of the tissue characteristic information comprises calculating the tissue characteristic information by using a lookup table in which tissue characteristic information corresponding to pre-diagnostic information is stored.

9. The focal point controlling method of claim 2, wherein the tissue characteristic information includes a propagation speed of an ultrasonic wave in a tissue and a density of the tissue, and
the controlling of the position at which the focal point is formed comprises calculating a propagation characteristic of the ultrasonic wave on the path by using the tissue characteristic information and calculating a particle velocity for generating the ultrasonic wave at a position at which the ultrasonic wave is generated to form the focal point at the target position by using the propagation characteristic.

10. The focal point controlling method of claim 9, wherein the propagation characteristic of the ultrasonic wave on the path is calculated by calculating a transmission coefficient, a characteristic impedance, and a wave number of each of the segments obtained from the path by using the tissue characteristic information.

11. The focal point controlling method of claim 2, further comprising segmenting the path through which the ultrasonic wave propagates into two or more segments based on the tissue characteristic information,
wherein the path through which the ultrasonic wave propagates is segmented by segmenting the path into segments having a same length, by segmenting the path according to a change in a computed tomography (CT) number of the tissues, or by segmenting the path according to a user's input.

12. A non-transitory computer-readable recording medium storing a computer-readable program for executing the focal point controlling method of claim 1.

13. A focused ultrasound therapy apparatus comprising:
an ultrasonic transducer to generate an ultrasonic wave by transducing an electric signal;
a pre-diagnostic image receiver to receive a pre-diagnostic image which includes tissue characteristic information of tissues on a path through which the ultrasonic wave propagates from a position from which the ultrasonic wave is generated to a target position on which a focal point is to be formed;
a tissue characteristic information acquisition unit to acquire, from the received pre-diagnostic image, the tissue characteristic information which includes physical characteristic values affecting the propagation of the ultrasonic wave; and
a focal point controller to control the ultrasonic transducer to form the focal point on the target position by using the acquired tissue characteristic information.

14. The focused ultrasound therapy apparatus of claim 13, wherein the tissue characteristic information acquisition unit acquires tissue characteristic information of each of two or more segments obtained from the path.

15. The focused ultrasound therapy apparatus of claim 14, wherein the tissue characteristic information acquisition unit comprises:
a pre-diagnostic information acquisition unit to acquire pre-diagnostic information from the received pre-diagnostic image;
a path segmentation unit to segment the path into a plurality of segments based on the acquired pre-diagnostic information;
a segment average value calculator to average pre-diagnostic information of each of the segments; and
a tissue characteristic information mapping unit to map the averaged pre-diagnostic information to tissue characteristic information by using a lookup table.

16. The focused ultrasound therapy apparatus of claim 14, wherein the tissue characteristic information acquisition unit comprises:
a pre-diagnostic information acquisition unit to acquire pre-diagnostic information from the received pre-diagnostic image;
an experimental linear model applying unit to calculate tissue characteristic information by applying the acquired pre-diagnostic information to an experimental linear model;
a path segmentation unit to segment the path into a plurality of segments based on the calculated tissue characteristic information; and
a segment average value calculator to average tissue characteristic information of each of the segments.

17. The focused ultrasound therapy apparatus of claim 15, wherein the tissue characteristic information includes a propagation speed of an ultrasonic wave in a tissue and density of the tissue, and
the focal point controller comprises:
a beamforming algorithm execution unit to calculate a propagation characteristic of the ultrasonic wave on the path by using the tissue characteristic information received from the tissue characteristic information acquisition unit and to calculate a particle velocity in the ultrasonic transducer to form the focal point at the target position by using the propagation characteristic; and
a particle velocity controller to control the ultrasonic transducer based on the calculated particle velocity.

18. The focused ultrasound therapy apparatus of claim 17, wherein the beamforming algorithm execution unit calculates a transmission coefficient, a characteristic impedance, and a wave number of each of the segments obtained from the path by using the tissue characteristic information received from tissue characteristic information acquisition unit and calculates a propagation characteristic of the ultrasonic wave on the path by using the transmission coefficient, the characteristic impedance, and the wave number of each of the segments.

19. The focused ultrasound therapy apparatus of claim 14, wherein the tissue characteristic information acquisition unit comprises a path segmentation unit to segment the path into a plurality of segments by segmenting the path into segments having a same length, by segmenting the path according to a change in a computed tomography (CT) number of the tissues, or by segmenting the path according to a user's input.

20. A focused ultrasound therapy apparatus comprising:
an ultrasonic transducer to generate an ultrasonic wave;
a tissue characteristic information acquisition unit to acquire tissue characteristic information of tissues on a path through which the ultrasonic wave propagates to a target position on which a focal point is to be formed;
a path segmentation unit to segment the path into a plurality of segments based on the acquired tissue characteristic information; and
a focal point controller to control the ultrasonic transducer to form a focal point on the target position by using the acquired tissue characteristic information for each of the plurality of segments.
